(19) United States
(12) Patent Application Publication
(10) Pub. No.: US 2018/0286500 A1
(43) Pub. Date: Oct. 4, 2018

(54) SYSTEM FOR ACQUISITION, PROCESSING AND VISUALIZATION OF CLINICAL DATA OF PATIENTS

(71) Applicant: Alberto SOLE GUERRA, Key Biscayne, FL (US)

(72) Inventor: Alberto SOLE GUERRA, Key Biscayne, FL (US)

(21) Appl. No.: 15/765,379

(22) PCT Filed: Dec. 31, 2015
(86) PCT No.: PCT/US2015/068216

§ 371 Date: Apr. 2, 2018

(51) Int. Cl. G16H 10/60; G16H 15/00

(52) U.S. Cl. CPC G16H 10/60; G16H 15/00

(57) ABSTRACT

A modular system and method able to carry out the following functions. Acquiring, standardizing and manipulating data obtained from any medical device, including digital data, waveforms, and administration of medication, images and lab results. Monitoring allowing customization of each view of the patient with his own characteristics and real-time visualization of included values: digital data, waveforms, video surveillance, medication and clinical milestones. Collecting information in a single powerful graphic user interface helping physicians to improve decision making and make clinical guides. Using statistical methods applied to real-time data helping to detect alerts and identifying physiological behavioral patterns. Connecting different medical devices to control the use of each device individually and transform the activity carried out in invoices.

### CROSS REFERENCE TO RELATED APPLICATION

This application is a national stage entry of PCT/US2015/068216 filed Dec. 31, 2015, under the International Convention.

### BACKGROUND

The present invention relates to the systems for acquisition, processing and visualization of clinical data of patients. The invention is based on the product “Middleware Device Interface M.D.I.” registered in the Intellectual Property, entry 09/2011/2442 in Valencia (Spain) as a computer software to the name of Alberto SOLÉ GUERRA (DNI 00827694-Q) and Sara ENRÍQUEZ ALONSO (DNI 45683444-Q), with request number V-2334-10 and filing date Dec. 29, 2010, 10:35 am.

### PURPOSE OF THE INVENTION

The first purpose of the present invention is a system for data acquisition from any medical electrical device, regardless of its manufacturer, type or model.

The second purpose of the present invention consists of a system to acquire, standardize, manage and visualize clinical data obtained from any medical or computer equipment in any sanitary technological environment, which allows physicians to assess and evaluate the health condition of the patient, in real time, increasing his safety and helping in medical decision making. This process consists of storing the structured data calculating the means by time ranges, managing the connections of the data sent by the devices, and making use of acquired data, displaying it visually by way of graphs and/or medical image viewers, creating customizable alarm systems per each patient, as well as exporting said information to third parties, using technological and sanitary standards. In addition, the system has a sophisticated integrated system to detect alerts related to standardized data. The aim of said second purpose of the present invention is to control clinical data on each patient in order to assess, both in real time and retrospectively (historically), the health condition and the current and future risks that may arise.

The third purpose of the present invention is a system making it possible to convert the data previously obtained from invoices related to the time and treatments used in a patient, and control the costs and consumption of resources of the means used.

### STATE OF THE ART

In a market of healthcare technology, often biased by heterogeneous, non-collaborative and non-interacting medical systems, the main actors (physicians, doctors, nurses . . . ) require efficient environments to avoid feeling overloaded using systems that consume excessive time, resources and efforts. Actually, the different IT solutions (EMR, PHR, EHR, PDMS, etc.) currently provided by manufacturers rely on a rigid and airtight system, based on specific and narrow views of clinical information. These approaches tend to be very rigid in terms of interoperability, resulting in poor interfaces between distinct sources of information and displaying little or absence of maneuver execution.

The Market of Healthcare IT is a complex, fragmented and heterogeneous ecosystem that needs to be integrated from data to decision support. There are three big areas of connectivity: the first was dominated by the big manufacturers and brands and it has its limitations, the second and current one is based on Medical Device Connectivity, Integrators and Medical Data Records and the future that will belong to the Era of Intelligence Data Mining.

Even amid recent healthcare reform acts in the U.S., healthcare spending as a percent of GDP is expected to increase to almost 20 percent by the year 2021. This is due to more insured individuals, increased home health spending, higher health insurance costs, and cost-sharing subsidies.

There are three strategies, all with similar goals and benefits to patients; they are used to control health costs at the consumer level:

Adopting wireless technologies in healthcare.

Upgrading and implementing advanced patient monitoring technologies.

Expanding the Telemonitoring reach and recognizing the cost control benefits.

Hospitals want to improve the level of care and efficiency at the same time because there are opportunities for both. There are approximately 100,000 deaths per year in the U.S. due to preventable medical errors. If hospitals have an opportunity to save costs and improve care at the same time they are generally more open to these systems. Additionally, prevention of medical errors is cost-saving in terms of reduced stay in the hospital, and malpractice or legal claims.

The case studies concerning patient monitoring technologies have described a high patient satisfaction ratio, a reduced readmission rate for recently discharged patients, and a level of increased efficiency for staff. Some of the most widely reported concerns from healthcare facilities involve standardization, data security, and future technology integration. The complexity of the healthcare system is providing opportunities in patient monitoring solutions in a wide range of specialties from emergency responders to chronic care providers. The global advanced patient monitoring market has seen some recent growth with the United States and many European countries at the forefront of implementation, so there is a need for Patient Clinical Data acquisition, analysis and monitoring.

Patent US 2014203937 as of 2014 Jun. 24 is dedicated to system to visualize clinical data through an interface divided into sections, so that statistics, waveforms, etc. can be shown in a section of the screen while applications can be launched from another section. However, it does not provide for the possibility of quantifying the time of use of each device, so it does not provide the possibility of billing or the calculation of costs. Likewise, the data visualizing method is very limited.

U.S. Pat. No. 8,645,164 as of 2014 Feb. 4 is dedicated to a system to visualize data received from different devices or apparatuses. It also includes database managing systems, among other aspects. U.S. Pat. No. 8,856,298 as of 2014 Oct. 7 is dedicated to a system to capture data from many sources, extract and present reports, including data standardization. All of them show the above said limitations: compatibility among different devices of any manufacturer is guaranteed, data visualization is poor or incomplete and it does not provide for the possibility of calculating costs or billing them.

U.S. Pat. No. 5,850,221 as of 1998 Dec. 15 is dedicated to a graphic interface system generating images that represent a treatment plan. Each order can be assigned a cost, the total cost of the different treatments can be determined. However, this calculation of costs is based on previous assignment to each treatment plan, the real use of each device or the means used not being evaluated, which causes a poor incomplete billing method, in addition to preventing effective control on the hours of use of each individual device.

But we can verify that there is important lack in the system used to date. The main inconvenience of the current state of the art is not data obtainment and classification but its presentation to the user, in this case the medical staff, in a single and homogeneous format, which requires data standardization into a common format. Likewise, the calculation of costs, with a dual purpose (control of costs and billing based on real use of the procedures used), has not been satisfactorily worked out so far. We verified that, although the references targeted at the use of systems to deal with clinical data are very numerous, those dedicated to comprehensively dealing with information are very scarce, including standardization of the different formats in which said data can be acquired and presentation of said information to the sanitary professional in a powerful unified format.

The present invention solves these defects by delivering a cost-effective and reliable alternative to the actual player's solutions. The invention can handle the care of patients in a multi facility environment allowing an easy way to follow up the patient across the overall process, in a non-intrusive method and low incidence on costs, delivering real outcomes that are translated into effectiveness and efficiency for the facilities, leaving all resolved satisfactorily with the innovative proposal submitted in this application solution.

### DISCLOSURE OF THE INVENTION

After studying many different systems and configurations, an innovative solution has been achieved, said solution being based on the sum of module elements that, in turn, are a combination of different components. Each of these components has a specific function, a highly flexible result being attained, its main achievements being as follows:

1. A method to acquire, standardize and manipulate the data obtained from any source of medical devices, including digital data, waveforms, administration of medications, medical images and lab results.

2. A monitoring method allowing customization of each view of the patient with his own characteristics and real-time visualization of the included values: digital data, waveforms, 3D patterns of Vital Signs, video surveillance, medication, alerts, diagnoses and clinical milestones. This monitoring method allows to view data in real time as well as historical data of the patient to follow-up the pathway and data across the time: clinical episodes, EMR, disease and others.

3. A method to collect valuable and relevant information in a single and powerful graphic user interface helping physicians to improve decision making and make clinical guides. The patient can be classified in different ways (trauma, cardiologic, breathing, renal, infectious, etc.) to be identified and have better control of related risks associated with the disease.

4. A method to use diverse statistical methods applied to real-time data helping to detect alerts and identifying physiological behavioral patterns in the course of time or during daily time phases (morning, afternoon, evening).

5. A method to connect different medical devices allowing control of the use of each device individually (based on the serial number) and convert the activity carried out into control of deterioration and/or amortization as well as in invoices of the services rendered to the patient.

The present invention; HIT Platform, aims to provide a smart solution that captures, standardizes, manages and visualizes the Clinical data acquired from any medical equipment, by integrating and consolidating their data independent of manufacturers and models. The invention of this disclosure uses a well-known standard catalogue to standardize the data (SNOMED, LOINC, DICOM, ICD9 and 10, Rosetta RTM and Private Catalogues) which, when used in combination with a scientific software process and GUI interface viewer component, is powerful for Medical Decision Support and user friendly, less expensive than previous MDC.

The solution is modular and allows solutions in isolated environments or specialized services as may be surgical environments, Intensive Medicine, Critical Care, Emergency, Resuscitation and could be scaled as a global technological solution for different facilities as Clinics, hospitals (plants and medical specialties) as well as health entities (Insurances, HMO, PPO, hospital groups) or governmental purposes (health regions RHIO or areas, NHS) covering a broad spectrum health population, allowing monitoring and exchange of relevant clinical data, focused on the patient.

HIT Platform can access the data of each machine identifying it by its serial number to satisfy:

Clinical issues through monitoring capabilities, alerts detected through out of range values, alarms identifying possible artifacts and anomalies, trend research, preventive dynamic medical algorithm system that prevent the detection of alerts.

Management capabilities making it possible to perform a real-time billing system per patient as well as control of the costs and concrete needs of the stock of medical devices in the facility.

Technology scope that allows a functional co-rapporteur verifiable system on the availability of medical devices including traceability and management by bioengineering staff.

This present invention is broadly concerned with the capability to capture all the relevant clinical information, manipulate this information and display it in one single view to give the professional teams (physicians, technicians and nurses) a fast, continuous and better health status of the patient to support in Medical Decisions, control and identify the diseases and significantly increase the efficiency of the clinical workflow process.

HIT Platform is composed of a suite of applications and services that can handle clinical information in order to obtain all the substantial data acquired from any Medical Device, using hardware components that will ensure connectivity and data transmission. This third-tier platform is a patient centered system with a multi-channel communication layer allowing visualization of data from diverse sources (Web Portals, Tablets, PC).

The Healthcare Interoperability Platform is based on 10 different modules grouped in three different categories: Connectivity and Data Manipulation, Clinical Viewers and Management and Configuration Tools. This suite of applications can perform the acquisition, storage, analysis, alert detection and visualization of any clinical data (Digital data, Waveforms, Medical Imaging, Lab results, etc.) of any patient.

### Category One: Connectivity and Data Manipulation

The core system of the Platform has two modules:

HIT Server(1): It runs in the central server that receives the standardized structured data, process it through different statistics and data algorithm tools.

HIT Core (2): It runs in the physical connector that is responsible for collecting the data every second or less in real time, structuring the data using a standardization process, creating average minute data and managing the alarm and alert data sent by the devices.

### HIT Server Module (1):

The Server Module of the platform stores and manipulates data in a local environment or in the cloud depending on the needs of each customer. The DB structure is based on an open source that can easily be scaled from small institutions to large communities or regions supporting clustering modes and load balancing capabilities in order to ensure access to the data. Data are structured into two different instances of the DB: the first one ensures the dynamic and real time data and the second one is the backup of the historical data that could sometimes be needed to be retrieved. The server Modules has different services that run at the level of the Operating System (supporting Windows, Unix or Linux) that supports the process on data on the following issues:

Means and Customized Average data defined in a master file per customer.

Statistical calculation methods for acquired digital data: Mean, Minimum and Maximum Values, Range, Standard Deviation, Variance Coefficient, Percentage of data out of Standard Deviation limits, BMI, BSA.

Alert detection System that generates the alert notifications associated when an abnormal or out of range data is captured and recorded per patient limits, Medical Device limits or Standards limits associated with patient typology criteria: weight, height, age, sex, BMI, disease, etc.

Artifact System Detection that looks at alarms sent from the medical devices, verifies with the actual alerts detected and recorded in the Database and specifies, in the structured data, the possible artifact correlative to the alarm received.

Gateway System that delivers all the data to mobile devices like tablets, smart phones and other devices in a secure way (encryption, SSL and private communication protocol) to ensure that any of them can store the sensible data outside the interface access session.

Seasonal Time Change (+1 h or −1 h) and GMT Date/Time control on stored data.

Rules on Deletion data process based on personalized criteria (time, type of data . . . ).
Load Balancing: HIT Database can be spread on multiple cluster using a Proprietary Load Balancing Mechanism. To create the load balancing, we designate One Main server on which we declare the list of clusters available for the rest of servers participating in the Load Balancing. One cluster is a server on which we install the database engine of HIT. Once declared on the Main server, a service will detect the new cluster and automatically deploy the structure and replicate the Main configuration data to get it ready to receive Patient data. Every time that a new patient is created, an internal trigger on the Main database will check the state and the capacity of each cluster and determine which cluster has more space available and then link the new patient to one specific cluster. After this distribution, all data related to this patient will automatically be sent to this specific cluster load balancing storage.

The server Module has also an EAI Enterprise Integration Engine based on messaging (XML, HL7 and Proprietary) and Web Services that will ensure interoperability with external services of the Entity like Laboratory, Blood Bank, Pharmacy, EMR and others to obtain relevant clinical data of the patient.

The server has an embedded and automatic updating mechanism to ensure the distribution of the versioning of the product and a watchdog that alerts the technical staff in case of process failures in the server or any detected physical problems (RAM, CPU, RAID system, etc.).

### HIT Core Module (2):

HIT Core is the software module that runs at the physical connector level. This module is a set of services and drivers responsible for collecting the data acquired from the Medical Devices. The service controls the Medical Device connection verifying that the corresponding driver is running in the system, detecting failures of communication and data transmission, as well as control of the standardization process that stores the real time data locally. It has a priority mechanism that controls which Medical Device has the highest sensibility and precision (defined by the entity) on the acquired measurement data and, during the standardization process, it stores only the most accurate data (which means that only one standardized data per patient is recorded in the local database using the best and most efficient Medical Device). It also has the ability to acquire the alarms sent from the Medical Device as well as any parameter or specific configuration that will be important for the Medical decision support like the Ventilation Modes from ventilators or the administration parameters like infusion rate, volume to be infused, time from the infusions pump.

This service is also responsible for creating the average data every minute and synchronizing with the previously described central server (HIT Server Module) and has the common issues with the services running at the server level:
Alert Detection System: that generates the alert notifications associated when an abnormal or out of range data is captured and recorded per patient limits, Medical Device limits or Standard limits associated with patient typology criteria: weight, height, age, sex, BMI, disease, etc.
Artifact System Detection: that looks at alarms sent from the medical devices, verifies with the actual alerts detected and recorded in the Database and specifies, in the structured data, the possible artifact correlative to the alarm received.
Seasonal Time Change: (+1 h or −1 h) and GMT Date/Time control on stored data.
Rules on Deletion Data Process: based on personalized criteria (time, type of data . . . ).
The set of Drivers stored in the hard drive of the HIT Physical connector can handle any data exchange protocol using standards (including IOT) or proprietary methods of communication (HL7, XML) (using separator, fixed length of data, structured data). The Driver can acquire any digital and analogical data (discrete or continuous like waveforms) that will be digitalized at the hardware components level from 0.01 second to 5 minutes capture frequency. The data transmission limitation is due to the restriction of the Medical Device or protocol created from the brand, HIT Platform's Drivers as a Machine Interpreter can handle and collect any data at any frequency and through any communication channel: Ethernet, Wi-Fi, Bluetooth, RS232, Analogical outputs, HDMI, NFS, USB, etc.

### Category Two: Clinical Viewers

HIT Platform's Clinical Viewers are the User Interface Modules that display all the information acquired and standardized from the Medical Devices as well as external Information Systems (Laboratory, EMR, Pharmacy . . . ).

The Clinical Viewers are five modules:
1. HIT Connect (3) is installed in the physical connector and displays data from the associated patient.
2. HIT Dashboard and Central Monitoring (4) is installed in any computer of the facility or entity that has access to the private network and the HIT Server carrying the information of any patient of the center.
3. HIT Telemonitoring (5) is tablet oriented software that delivers a Home Care Telemonitoring system to follow up the health status of patients anywhere and the last status.
4. HIT Clinical Reports (6) is software that creates reports based on specific criteria in order to share with external systems like Medical Records, PHR and EHR.
5. HIT Advanced Clinical Dashboard (7) is a software installed in computers from Physician and Nurses central station where they can review any historical data of the patient independently of the place or service, the time or the prior episodes, and visits made in the medical entity.

These five modules allow management of the acquired data locally, remotely and centrally, to visually present the information through charts and graphics (GANTTs representation, viewers of medical images DICOM) and export the information to third parties, using standard technological formats (XML, Excel, Word), which are described below:

### HIT Connect Module (3):

This software module presents the clinical data from the Medical Devices connected to the patient. The main purpose of this module is to permit the professional staff to connect and associate the Medical Devices to the patient and view the acquired real time data and review the trends and average data of the recent past (last 1 h to 24 h). The connectivity of the specific (using SN bar code system based in QR codes) Medical Device is a very simple easy-to-use mechanism that has a visual interface that represents the devices per group of devices with their specific status (localized to physically identify the place where the Medical Device is, connected when it is connected to HIT Connector but not acquiring data, and communicating data when we standardize and process the data).

HIT Connect delivers, in chart view and table format view, all the standardized data including detected Alarms and Alerts, laboratory results, Medical Imaging, Clinical Events and Drug administration. Data capture is associated in different customized anatomical groups (Vital Signs, Ventilation, Medication, Hemodynamic, etc.) that are represented in one single chart with the possibility of defining the measurements that should be graphed. We can also view the waveforms in real time or during a specific period as well as all the related parameters and configurations donated by the Medical Devices that indicate their status (e.g. Infusion rate, Therapy and VTBI for Infusion Pumps).

This module can also access the Demographic, anthropometric and Clinical History (Backgrounds, Allergies and Alert Personalized Limits) data of the patient that can be introduced manually from the interface or integrated from external Systems (EMR, EHR, PHR).

### HIT Dashboard and Central Monitoring Module (4):

The Dashboard and Central Monitoring Software are focused to allow the medical team of the entity to access any Clinical data of any patient. This module has much common functionality with the previous HIT Connect regarding the chart, graph and viewing patient data options. However, from the Dashboard and Central Monitoring we can have access to the list of patients in a table view and cartographic view of any floor, service, area or unit (bed, box, Operating Room) of the entity, facility, region . . . . Also, for the Home Care Patients we can monitor, in a geographical view in real time, the Telemonitoring of the different devices that are connected to the patient in a continuous and discontinuous mode.

The main difference of the charting viewer system of the previous module is mainly that you can open several instances of the same patient or different patients at the same time and study the graphs among different timeslots and all the charts are represented in an embedded display per anatomical groups that could be viewed in a tabular or mosaic view (all at the same time in different and parallel windows). Any of the measurements could be graphically displayed in the separate chart per anatomical group or a chart comparison can be created on demand with any of them. One big difference between the charting tool and the 3D graph is that the 3D graph can represent data in 3 axes: the first one could be one-hour display or 24 hours depending on the range of selected dates, the second axe will represent the number of hours (up to 24 h) or the number of days and the third will represent the axe of values of the selected measurement. This could help physicians to identify patterns of vital signs or even identify related risks in a timing periodicity related to drug administration, sleeping times, cleaning activities or other possible reasons. This 3D chart system could be easily used in the comparative view from any graph we selected.

For all these charting tools we can modify the type of line, dot, color, graph representation, add notes and Region of Interest in the chart, export the actual view and zoom in and out. All the charts can be displayed in one single Y axe or multiple Y axes associated each one with the Unit of the graph or measurements. We also have an auto-refresh mechanism that gives the end user the possibility of specifying, from 1 minute to 60 minutes, the refresh time of the overall context choose (measurements, time range, axe view) assuring continuous refresh of the information even if the end user is not be present anymore.

The Central Monitoring module can show, in real time, the data collected from any medical device connected to any patient, selected independently of his location, with the following views: Wave Forms, Digital Data, Cartographic View, Video Monitoring, Clinical Events, and Dashboard View where we can mix any Clinical real and non-real time Data (DICOM Images, Lab results, Wave forms, Values, Medication, History and Background). The instances of the Central Monitoring can be running at the same time and this does not have any limitation. All the displayed data are built in a widget technology that can be modified separately in weight, height and position.

### HIT Telemonitoring Module (5):

The Telemonitoring software is a mobile technology based on iOS and Android platforms that have the ability to follow up patients in a table and geographic view based on Google Map Mashup technology. The entire single patient monitoring data could be retrieved in real time and in a trend format with continuous and non-continuous mode including management of alarms and alerts detected by the HIT Telemonitoring Connector. The Telemonitoring module also allows the possibility also of selecting the measurements to display, having access to the medication, medical images, and establishing a direct video-conference session with the patient to assist and communicate health recommendations to improve the security at home environment. All Clinical Data could be accessed including background, allergies, patient history and other relevant issues. The drug management functionality acts as an eMAR (electronic Medication Administration record) that can be followed up remotely by a nurse at the entity level (Hospital, Clinical, facility). This module can be integrated for health transport via ambulances, helicopters and boats using the LTE capabilities of the HIT Telemonitoring connector, in order to transmit all the real time data, including scales evaluation like GLASCOW to improve the triage at the ER, to the Emergency rooms of the hospital/clinic where the patient should be admitted.

### HIT Clinical Reports Module (6):

HIT Clinical reports are composed of a set of modules integrated in the HIT Connect, HIT Dashboard and Central Monitoring interfaces, which handle the collected information in a certain period of time and create automatic information reports with a high clinical data content that can be transmitted using the HIT Server EAI functionality to the EMR, EHR, PHR system that is implemented at the facility. All the information is exported into universal formats: PDF, WORD, EXCEL, XML.

HIT Clinical reports are based on the following Clinical Documentation:

Evolutionary Clinical Report: based on daily or weekly resume information, we create the trends and important milestones in a visual report (Charts, Tables and Notes).

Customized View Report: this report is complete user tailored information built according to the chosen criteria (dates, measurements, values, etc.).

Patient File Report: the complete resume information on the patient from one or more episodes that is very useful for the final discharge letter.

Drug Administration Report: indicates the medication delivered, administrated and refused per patient or group of patients including infusion pump administration, manual, micro drip, syringe, etc.

Global reports per patient with worst and best results/outcomes achieved.

### HIT Advanced Clinical Dashboard Module (7):

Advanced Clinical Dashboard allows to retrieve the historical information of the patient and present it in a single screen where the doctor can navigate chronologically through the different episodes and services where the patient was attended. All the data recovered from medical devices and information systems such as laboratories, Electronic Medical Record or image can be represented graphically or in a tabular format. This data helps medical decision support to identify problems in a specific time or through the different services that today didn't communicate that information and display it in a single dashboard. Historical data could be used in multi-episode patients to evaluate the follow-up and progress of the patient or the evolution of diseases (chronic, elevated risk diseases) or procedures (Surgery, rehabilitation, long term treatments . . . ).

Advanced Clinical Dashboard has the possibility of dynamically visualizing the development of the patient's condition in a movie format using a Clinical Data Player using a “play” mode button that allows to see the evolution of the patient as if it were a film that contains all the patient's data and can be easily review without having any User intervention. At any moment you can stop, rewind or go forward in the Clinical Data Movie to review or skip some data information. All the contents are updated and displayed in the Advanced Clinical Dashboard dynamically including: Vital Sign Trends, Medication, infusions and bolus, In & Out fluids, Clinical Events, Alarms and Alerts, lab results, Medical Imaging, Medical Annotations and risks based in Predictive analytics. The system can be configured using a graphical tool to select the specific period of progress during the Clinical Data Player. All the clinical events are marked in the Vital Sign Trends in order to identify it and can be compared with the patient status evolution based in the charts.

Advanced Clinical Dashboard has specific tools to zoom in a period of time and deliver a more detailed view of the information (all the above mentioned) including statistical information (average, minimums, maximum, standard deviation, Variance Coefficient, Percentage of data out of Standard Deviation limits), waveforms in a graphical view or tabulated view of that period. This module has a complete Mathematical tool library to make measurements and retrieve qualitative data from the trends using min/maximum functions, time/frequency and amplitude signals inside waveforms and can create dynamic formulas to be represented in a graphical chart or a table value format.

### Category Three: Management and Configuration Tools

The Management and Configuration system of the Platform has four modules associated in two different groups:

The first one is HIT Management Group that controls the activity of the locations of the entity.

The second is the HIT Configuration Group used to define the customization of the business rules of the platform.

### HIT Management Group:

This group is composed of three different modules: HIT Billing (8) that supports the control of the Medical Devices used per patient and generates the invoice corresponding with this usage, HIT Management (9) mainly focuses in statistics and Business Intelligence Analysis and, finally, HIT MD (10) responsible for the fleet of the Medical Devices.

### HIT Billing Module (8):

This financial module creates a bridge directly with the accounting system of the Entity using its own billing codes and generates an automatic invoice with the exact amount consumed by the patient including the administrated drug, the time of usage of the Device and the number of sensors or modules used from the Medical Devices according to specific rules associated per Brand and Model that define the amortization cost of each individual device.

All invoices have a Bar Code System (QR Codes) that identifies the episode or period of billing and can be easily retrieved from the platform by reading the BC in the billing/episode searching engine of the platform at the GUI interface level.

This Module can define the business rules to fix the cost units mapped with the internal accounting codes and can be crossed with a CPT standard nomenclature.

Finally, we can generate Global Billing History and reports that can be used for financial and outcome measurement purposes.

All the information will be sent to the accounting system of the entity by using the EAI engine of the platform in standard formats like XML, EDI and others.

### HIT Management Module (9):

HIT Management module helps decision makers and managers to have better control of the activity related to the usage of Medical Devices and professional staff inside the company. This module manipulates the real time and offline data in order to provide statistics and a Business Intelligence Dashboard based on ROLAP information cubes that can filter and display the information based on temporal criteria (day, week, month, trimester, semester and yearly) and activity data like number of beds, patients, medical devices, medications, measurements read, etc. This helps the managers of the company to find the real-time costs and profits of the activity very efficiently and export to universal document formats (XML, PDF, EXCEL, WORD and PowerPoint) that can be used externally or imported into external Information Systems.

### HIT MD Module (10):

HIT MD delivers a Real Time statistics in a Dashboard format of the fleet of Medical Devices with a Business Intelligent interface based on ROLAP information cubes that handle the usage and status of each device per brand, model, group, etc. Through this module we also can create and identify new Medical Devices, update the status of those that were reported as broken or unusable, print the Bar Codes associated or deleted from the database if needed. This module is targeted at the bio-engineering staffs of the company who will need to have a detailed control on the devices and it can create reports and statistics for the managers using the export universal engine of document format (XML, PDF, EXCEL, WORD and PowerPoint).

### HIT Configuration Group:

This group is composed of only one module: HIT Configuration (10) that has access to the configuration tables of the platform to perform changes on the different parameters and business rules. The main functionalities and data manipulation of this module are described on the list below:

### HIT Configuration Module (11):

Multi-Environment Management Tables: make it possible to create, modify or delete the hospital(s), services, areas, units (bed, boxes) of the facility as well as configure the basic parameters of the entity. Management and edition of the cartographic tool.
Platform System Management: can handle the Users, Profiles and grant access, HIT Connectors and servers installed.
Configuration and Defaults Values: of HIT Platform
Catalogue and Master Files Management including healthcare standards (SNOMED, LOINC, DICOM, ICD9 and 10, Rosetta RTM, etc.)
Language Content Manager: using 4 active languages at the interface and contents simultaneously and up to 7 different languages at the user interface (English, Spanish, French, German, Chinese, Japanese and Brazilian) with the possibility of adding up to 64 different languages.
Editor for the Preventive Data Analysis based on specific criteria using simple conditions operations to detect and notify possible alerts and organ failure risks.
Installation configuration Manager: Every HIT Connector are configured to communicate automatically with the Application Server. Every 30 seconds, each of them will send a request to the Application Server to check if it has any task to execute. We are using dynamic compilation in order to send potentially any kind of task. It could go from Installing or updating a service, checking or updating database datas to restart the HIT Connector, checking the status of the drivers, etc. . . . . This way we are able to manage fully every HIT Connector from the Application Server. We can deploy entirely a new HIT Connector without any needs of direct connection to it. Once a new HIT Connector is plugged on the hospital network, it becomes automatically available on the Application Server and ready for communication. On the Application Server we can monitor all the HIT Connector available, we can see which one is active, which one is sending Patient data, which one in synchronized or not, etc. . . . .
Personalized Washdog system: HIT contain an intelligent Washdog Dashboard to follow-up in real time the activity of any Medical Device Connector and identify and notify communication errors, physical errors or syntactic errors associated with the data received in case of data emission corrupted. From the Washdog we can intervein in the system and send specific commands to the connectors to stop, reload, restart or start specific services of HIT to assure the data integration and Medical Device Connectivity.

### Advantages Regarding Previous Systems:

The invention shown herein is an innovation the structural and constituent characteristics of which are unknown so far for the purpose to which it is intended, since it has the following advantages regarding previous systems; these reasons, in addition to its practical usefulness, endow it with enough grounds to obtain the exclusivity privilege that is requested:

### HIT Server Module (1):

Unlike other market technologies (Central Monitoring), this module makes it possible to manipulate data acquired from any electro medical device. This module is focused on the functionality of use and intelligence of data taken from any clinical database regardless of its manufacturer and model (the big brands only deal with their own apparatuses). The main competitive characteristics of this module are as follows:

Calculate means on the captured values. Said means are generated upon user customization. The other technologies only store gross data. Make statistics not only on acquired data but also on the means to assess trends.

A single system allowing automatic correction of correlative data caused by external artifacts. Typically, when a zero occurs in a monitor or when a sensor such as SpO2 is extracted, the acquired data is registered but, with the present invention, it is discarded as it is an artifact.

Alert detection system based on user defined criteria and not on ranges, as the other current systems do.

Management of time changes for data storage. Currently, in the market, these data can be discarded. We also included the possibility of having a geographic/hourly trace from where the data was taken in the case of being implemented, for instance, in a country where there are several time zones.

Customized data erasing system according to clinical criteria defined by the user, not by the technology.

### HIT Core Module (2):

This module has a so far unique component: the data standardizing method (analogical, digital, waveforms, images, videos . . . ). This method consists of managing a private (defined by each user) or public (based on a standard) catalog where the data from electro medical devices or clinical information sources such as Laboratories, Medical Image, Microbiology, Blood Bank and others will be crossed and mapped. The advantage of this module is also the means per minute which apply only to data validated by the intelligence of the artifact detecting system and priorities for data acquisition (for instance, if there are a monitor and a ventilator connected, only the breathing rhythm of the ventilator will be taken into account as they are more reliable sensors and, in case the ventilator is disconnected, the monitor will automatically continue reading said measurement, the source of said entry being identified in both data). This process is unique and is the differentiating foundation of our system. In addition, it not only reads data from the device but also univocally identifies them (by serial number), which makes it possible to identify any physical failures or systematic reading failures per unit of device. The system applies the same customized alert detection algorithm as the module described above. Another characteristic of this module is that it is independent of hardware and can be applied to any computer equipment (including tablets).

### HIT Connect Module (3):

The modules of the category Clinical Viewers are authentic Clinical Control Panels and not simple clinical information managers such as EMR (Electronic Medical Records), PDMS (Patient Data Management Systems) and others. In fact, our technology has been thought so that our viewers can be easily integrated into other interfaces through portable web technology and/or objects (Active X or NET Component or Applets Java).

Detailed evolutionary view according to time ranges, showing calculated values such as means and trends.

When far data are zoomed (+12 h before), the granularity of data is widened according to the selected zooming time: for instance, if in the view of 12 h, the data on 15-minute means are shown and a zoom corresponding to 2 h is made, the shown data will be data on 5-minute means and, if the zoom gets close to 1 h, data up to minute or second means can be shown.

Simultaneous view of clinical data such as Vital Signs and administration of medication through infusion pumps or automatic medical administration systems to correlate the effect of drugs or medicaments with the body reaction according to the administered volume or dose. This point is crucial, since no invention to date collates, at the level of interface, said information which is one of the first ones the medical staff collate on their minds.

Control, from the interface, of the devices connected to the patient, with the possibility of controlling several conditions (in the case of not wanting to take data): localized (it makes it possible to know where it is located), connected (it has a connection with the connector) and capturing (it is in the process of transmitting clinical data).

Alarm visualization system in many representation formats (Graphs, tables, Gantt), it is possible to define, for each of them, the classification to which it belongs (defined by the user and/or client) and the affected organ(s) in order to identify behavioral patterns (when an alert of the type of cardiac output is generated, then an alert of the type of breathing failure is chained later) in types of patients (cardiologic, breathing, renal, etc.). This logic can be taken to the level of medical praxis but not in management and data capture, so nurses usually have to turn off the alarms of the devices but they do not relate them among them unless the patient gets worse, when many times their sequence predicts which the evolution may be. This is collated in our preventive system based on analysis of data and detected alerts.

### HIT Dashboard and Central Monitoring Module (4):

Below is a description of the differences with other previous systems:
The Central Monitoring is an individual set of monitoring of each patient, it is possible to manage each one differently (the existing ones work with the same data for all patients). Besides, being based on widgets, it can make a very customized comparison of the monitoring interface and open many simultaneously (over 128 patients can be simultaneously monitored with 8 screens).
A very important aspect is that it includes real-time monitoring of the administration of medication through pumps.
Visualization of 3D clinical data. Longitudinal graphs (of 3 days, for instance) are composed in tridimensional graphs with cuts of 24 h or one hour in axis X, the days or hours in axis Y and the value(s) of the constant(s) in axis Z to assess the long-term behavioral patterns. For instance, it is possible to detect that there are biological changes (Blood Pressure, SpO2, Temperature) every night from 12 am to 2 am that can be caused by many factors: effects of medication, beginning of REM sleep, change of position, etc. In this case, a valley crossing said hours in said measurements would be detected.
The system can monitor not only inpatients but also patients at home (home hospitalization, for instance).
Access to the record of each patient from the Central Monitoring itself, making it possible to see trends, clinical data, alerts, etc.

### HIT Telemonitoring Module (5):

This module makes it possible to extend the technology to any mobile device (telephones, tablets, PDA, etc.).

### HIT Clinical Reports Module (6):

This module has a value of extension to the clinical reports and technology that can be exported and added to the HCE or other information systems. It generates evolutionary reports that can be differentiated by the use of data on trends (means) and sampling of administration of medication with detection of alerts based on the criteria and inventions of the previous modules.

### HIT Advanced Clinical Dashboard Module (7):

This module is unique as it delivers a chronological and historical summary view of the patient independently of the service, entity or procedure(surgery) executed during the different patient's episodes. On top of that, Module 7 contains an advanced Clinical Data Player permitting to review the information dynamically presented as a Patient Movie Disease progress. Advanced Clinical Dashboard could be used for different purposes like: Patient Follow-up, Disease Controls, Risk management and assessment, Clinical Data Interoperability and Scientific studies or Medical Investigations using the mathematical library contained.

### HIT Billing Module (8):

This module is unique since nobody currently crosses the time of use of devices with the real cost per measurement unit (sensors used, modules of the device used, medication and running time of the infusion pump, etc.) of each of them and it makes it possible to generate a use-adjusted invoice that can be integrated into the billing system of the entity, using its billing codes, also enabling assessment of the amortizing times of electro medical equipment in the entity. Currently, this is a big defect of management in hospitals, clinics, and specialized centers that have minimum control of the equipment used.

### HIT Management Module (9):

Likewise, this module is a development of an inventive nature. To date, no references on control of data on use of devices are known as nobody records the activity per unit of device (let us remind that our invention identifies the piece of equipment by its serial number and there is total control of its location, use and functionality). This makes it possible to have a statistical database of use by manufacturer, models, types, etc. Said use is most valuable regarding expenditure on electro medical equipment in the entity.

### HIT MD Module (10):

This module makes it possible to provide bioengineering or those responsible for the stock of medical equipment with a unique tool to control, identify and record the mishaps and problems detected in each of them. This module is intrinsically related to the previous one, the former is targeted at management and expenses and this one to identification, control and technical support.

### BRIEF DESCRIPTION OF THE FIGURES

In order to adequately illustrate the present invention and help to have better understanding of its fundamental characteristics, the present descriptive report is accompanied, as an integrating part of it, by the following figures of an illustrative non-limiting nature.

FIG. 1 represents the icons of those elements of the invention that are involved, in the case of a mobile unit, in direct communication with a hospital facility;

FIG. 2 shows the screen capture of the external device Philips IntelliVue MX800 through the connection screen HIT for the case of FIG. 1;

FIG. 3 shows the latest trends of the waveforms of the patient in real time on the screen of the mobile PC for the case of FIG. 1;

FIG. 4 shows on the screen information on mean values and trends received from Phillips Monitor since the beginning of the collection of data for the case of FIG. 1;

FIG. 5 shows on the screen the infused volume, the dosage and speed of infusion of the administered medication as recorded in the database for the case of FIG. 1;

FIG. 6: shows on the screen the Advanced Clinical Dashboard for a patient with historical data including all the trends, real time Monitoring, lab results, In & Out, Medication, clinical Events, Alarms and Alerts and History Pathway Summary as recorded in the database for the case of FIG. 1;

FIG. 7: shows on the screen the Advanced Clinical Dashboard detailed information for a patient including all the trends, clinical events, lab results, In & Out, Medication, Statistical data and Mathematical data calculated on waveforms as recorded in the database for the case of FIG. 1;

FIG. 8: shows on the screen the HIT Connect Dashboard detailed information for a specific patient for his real time data including all the trends, monitoring real time data, waveforms, lab results, In & Out, Medication, Alarms and Alerts, Clinical Events and Medical Image as recorded in the database for the case of FIG. 1;

FIG. 9 shows a general view of the process of a Mobile Unit with Direct Communication with a Facility;

FIG. 10 shows a flowchart of a Clinical Case report;

FIG. 11 shows a screenshot of a HIT screen;

FIG. 12 shows a screenshot of the waveform trends of the last 10 minutes in the display of the mobile PC;

FIG. 13 shows a screenshot of the trends and average data received from the Philips Monitor;

FIG. 14 shows a screenshot of a Medication screen from the command panel of HIT;

FIG. 15 shows a flowchart of the Telemonitoring System from a Clinic and the Patient's home with Physician's tablet (iPad, Android, Windows 8) activity remote control.

FIG. 16 shows a screenshot of a HIT Dashboard module showing a geographic view of the city with the clinic and the patients who are being monitored at home;

FIG. 17 shows a screenshot of the real-time data capture of data obtain via the standardization process and transmitted by way of 3G communication to the database that is installed in the clinic;

FIG. 18 shows a screenshot of the screen to gain access through a portable tablet that has an LTE connection allowing access to the information on a patient;

FIGS. 19 to 22 show a screenshot of the HIT Telemonitoring module of the patient;

FIG. 23 shows a flow chart of a Central Monitoring of a PACU unit with discharge of one of the patients;

FIG. 24 shows a screenshot of the HIT Dashboard module to follow up the patients hospitalized at the PICU service;

FIG. 25 shows a screenshot of a pop-up window of the HIT dashboard module of FIG. 24;

FIG. 26 shows a screenshot of the central screen of HIT Dashboard module;

FIG. 27 shows a screenshot of the Central Monitoring screen for the patient; and

FIG. 28 shows a screenshot of the Central Monitoring screen for the patient showing the discharge in order to recap all data on the patient including the invoice for the expenses and use of all the material he has been applied.

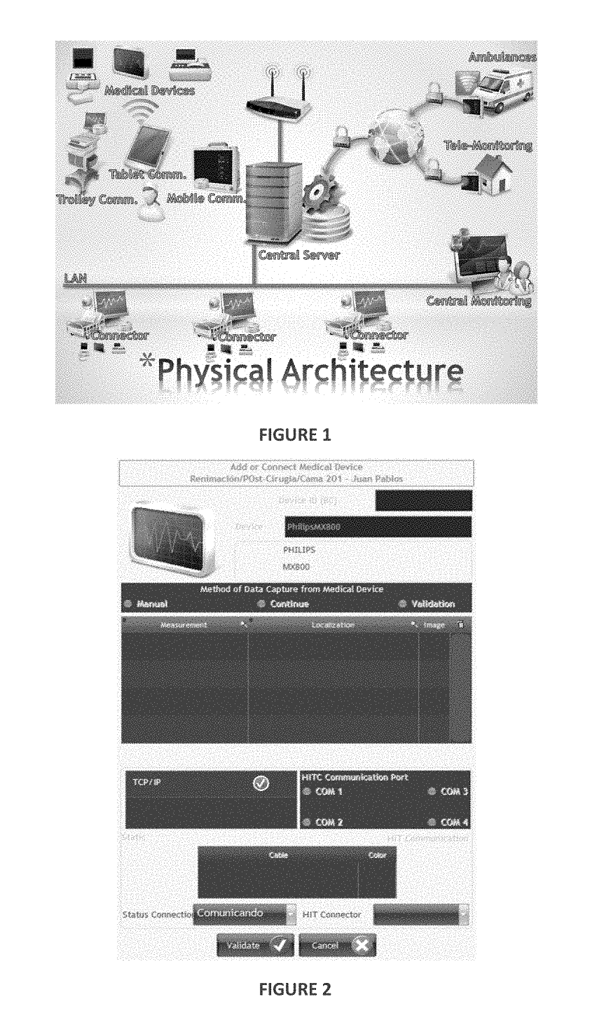

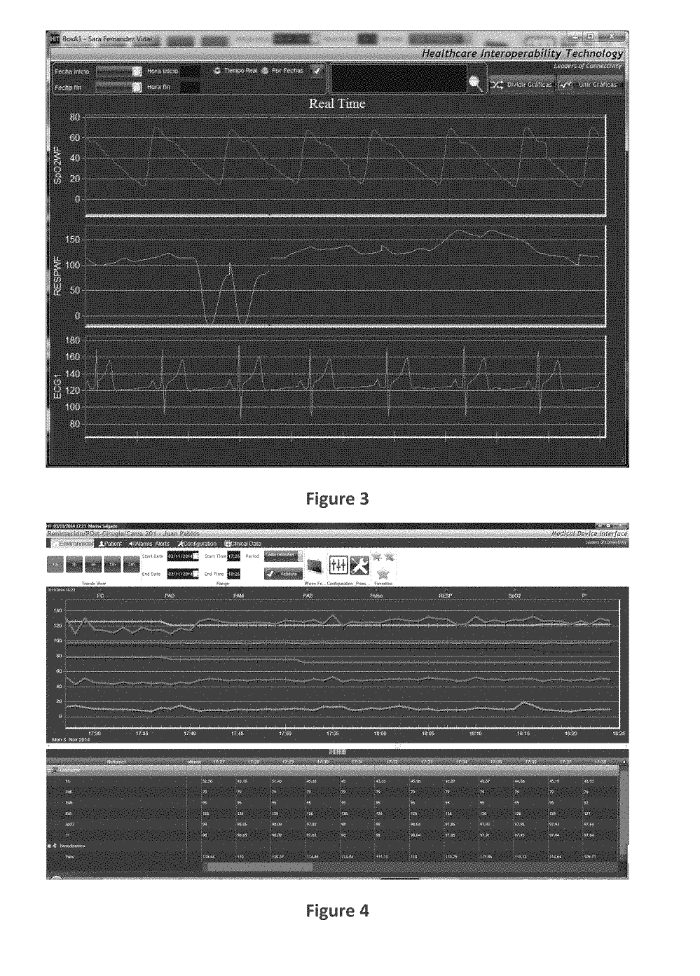

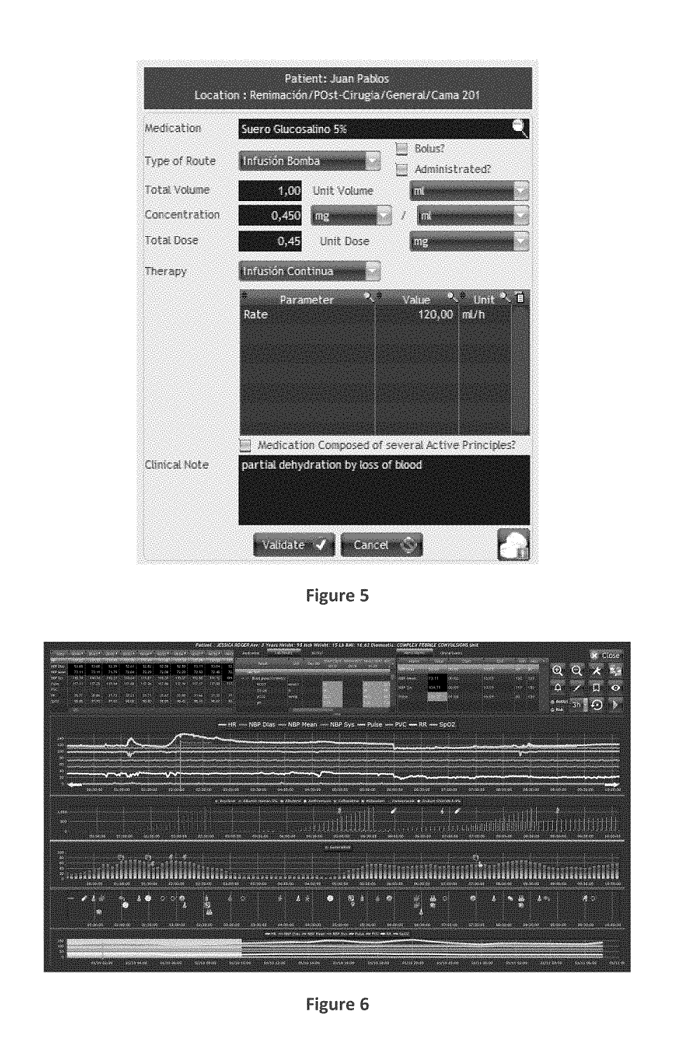

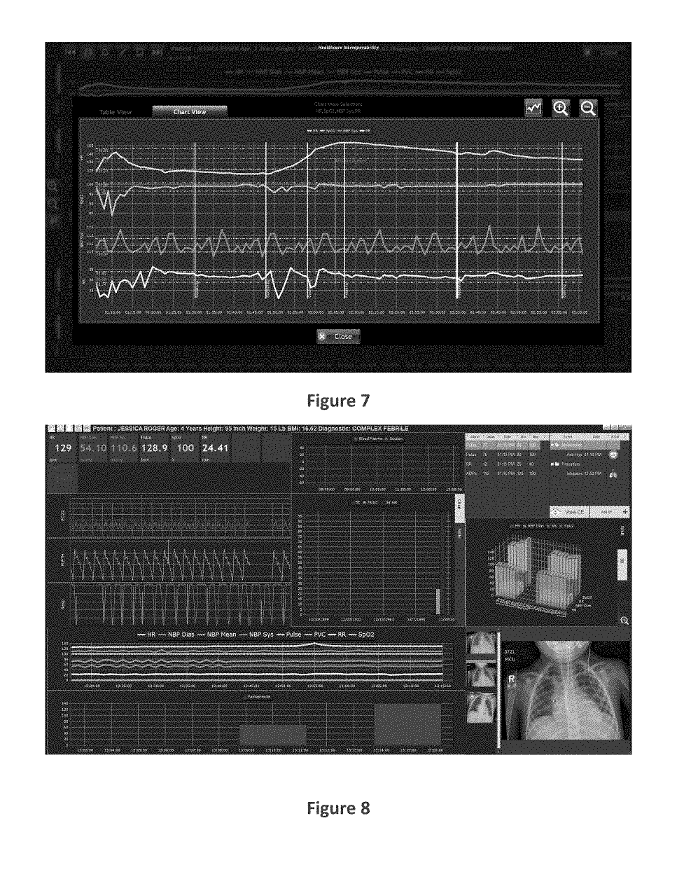

### DESCRIPTION OF THE PREFERRED EMBODIMENTS OF THE INVENTION

The system is based on the sum of elements, each of which has a specific function. Below are several examples of embodiments of the purpose of the invention, showing their functions in the different cases.

### Embodiment 1: General Description of the System of the Present Invention

Typically, HIT platform is installed in one or several services, in a hospital, clinic or any medical facility with one HIT Connector per each patient, continuous monitoring required (Intensive Care, OR, Emergency) and a number of HIT Trolley Connectors to asynchronously monitor the patients in the other beds or units. All the data acquired from the different units are stored in a central server that processes the different operations and algorithms to complete data manipulation. From the different PC of the facility, the medical staff can check, review or add clinical relevant information on the status of the patient. Outside the walls of the hospital or Clinic, HIT Platform provides a connectivity solution for the healthcare transportation companies and Telemonitoring programs ensuring continuous Telemonitoring and care of the discharged or pre-admitted patient. All the data collected during the different episodes are stored in the History instance of the central Database and can be used for statistical, research purposes or patient background check.

### Embodiment 2: Mobile Unit with Direct Communication with a Facility

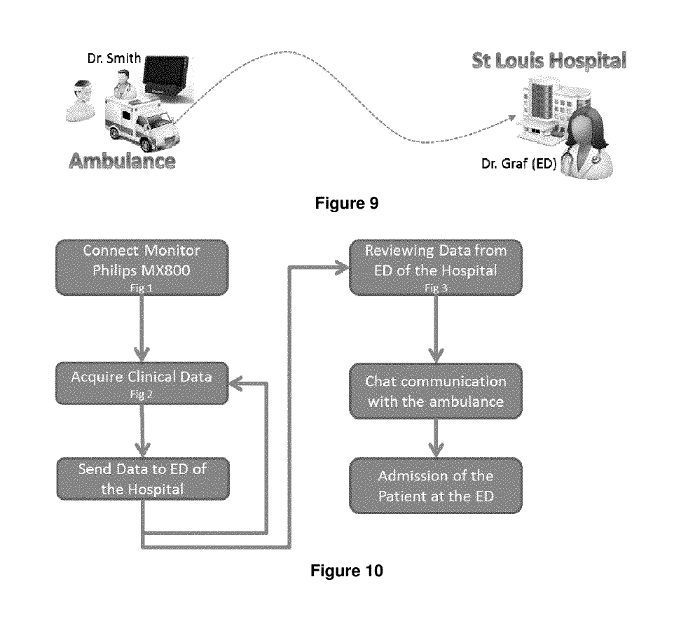

A general view of the process is represented in FIG. 9.

Clinical Case report of casualty has to be rushed to St Louis Hospital. The ambulance physician; Dr. Smith, assesses the patient's health condition at the place of the accident and decides to move to St Louis emergency hospital. The flowchart is shown in FIG. 10.

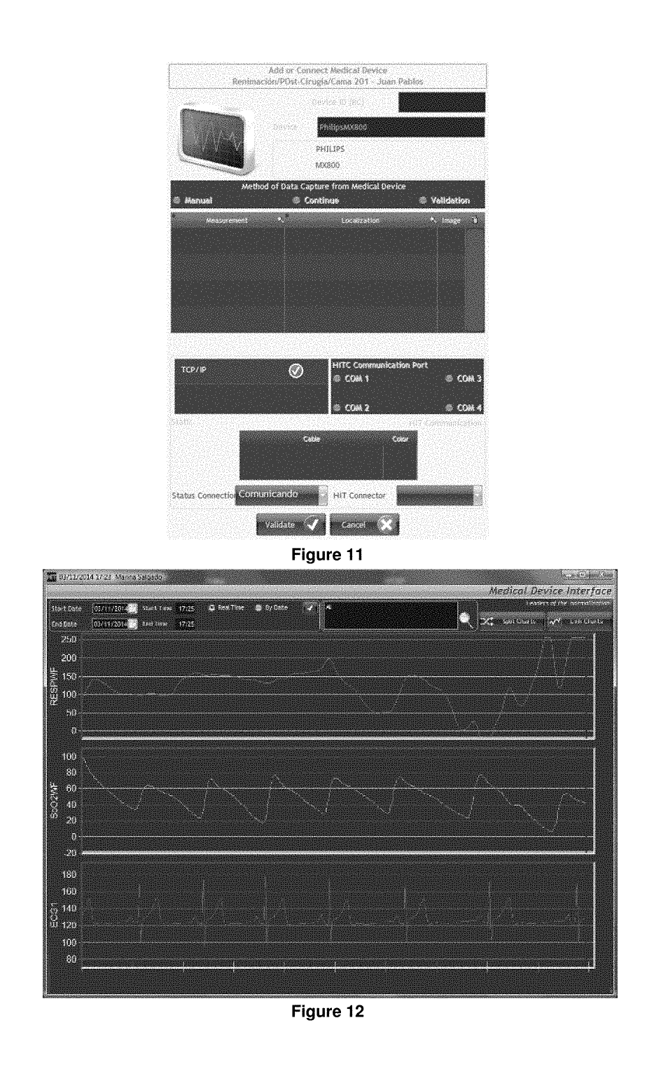

The doctor connects the patient to a Philips IntelliVue MX800 laptop monitor to monitor Blood Pressure, heart rate, SpO2 and continuous 12-lead electro to the Medical Device Connector of the Ambulance. The process is as follows: the doctor identifies and enters the demographic data of the patient at the mobile PC using HIT Connect software. Then he selects from the HIT connection screen (see FIG. 11) the Monitor Philips Intellivue MX800 (Medical Device) by reading the Bar Code in a QR format that will identify the Model and Device by its Serial Number. The doctor needs to specify the method of data capture from manual (to be entered manually), continue (in real time using the export data exchange protocol of the medical device) or validation (the captured data is sent to a validation interface that will validate each data sent before being stored in the HIT database). The Doctor will start the process of capturing and normalizing data by selecting the status “communicating” and validating the connection with the Monitor using the validate button. As soon as he starts the acquisition process, the HIT Core service installed in the mobile PC will look at the master tables where it will identify the Monitor Device, read the parameters and measurements available for this device (NBP, HR, SpO2, RR and 12 ECG), look in the table that maps every value with the normalized values (SBP: Systolic Blood Pressure, DBP: Diastolic Blood Pressure, MBP: Medium Blood Pressure, HR: Heart Rate, SpO2: SpO2, RR: Respiration Rate, RR WF: Respiration Wave Form, ECG I, ECGII, ECG V) of the HIT catalogue and look if there is another connected device that is using the same normalized values in order to detect which will have the highest priority for the data that should be stored in the database (in our case, none). The HIT core will start to acquire the data from the Philips monitor, stored in the local database and send the information using an LTE communication to the server in the cloud which can be viewed in real time from the Unit at St. Louis Emergency Hospital.

The doctor on the ambulance follows up the status of the patient and reviews the waveform trends of the last 10 minutes in the display of the mobile PC as shown in FIG. 12.

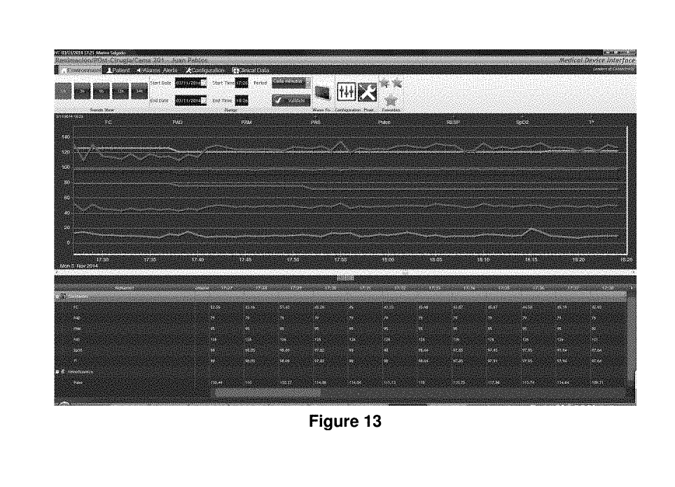

The acquisition process is executed every second and the stored data is sent progressively to the ED of St. Louis Hospital every minute in an average data process. From the ED, Dr. Graf is reviewing the information received from the beginning of the process that belongs to the trends and average data received from the Philips Monitor (show in FIG. 13); she evaluates the unstable situation of the patient due to SBP, HR, RR and SpO2 irregular values received and viewed in the charts.

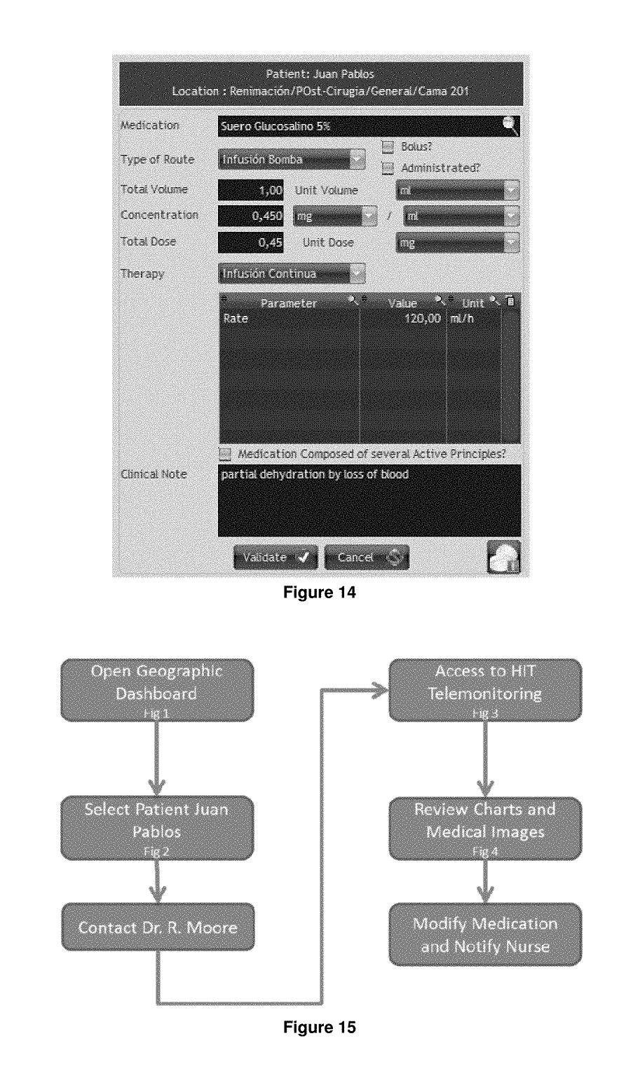

Dr. Graf decides to open a chat session with Dr. Smith using the HIT functionality of chat communication with external parties and indicates him that the patient lost a lot of blood and he should administrate a 5% serum to hydrate the patient before he arrives at the Emergency Room. Dr. Smith at the ambulance starts the process of administrating the medication indicated by Dr. Graf. He selects the add Medication screen from the command panel of HIT and looks for the 5% Serum that should be administrated manually using a micro drip. Dr Smith needs to specify the route (Infusion Pump), the volume to be infused, the dose and the infusion rate to apply in order to register this administration in the database (see FIG. 14). This administration is followed from the ED of St Louis Hospital and the outcome on the patient's status is checked in real time with the HIT Telemonitoring capabilities. This will also help the triage when the patient arrives at the Emergency room and will provide better and more accurate information about what was happened during the transportation of the patient.

Through central monitoring, emergency physicians can view, evaluate and chat in real time with the doctor on the ambulance to have exhaustive monitoring of the patient during transport to hospital. The patient triage is performed naturally and quickly in the Emergency service, which expedites care of the patient and prepares for the necessary medical material.

### Embodiment 3: Telemonitoring System from a Clinic and the Patient's Home with Physician's Tablet (iPad, Android, Windows 8) Activity Remote Control

At Valier Clinic in Jacksonville, Fla., patients are monitored at home. They can be monitored from the entity through the Central Monitoring of the product, the activity of the patient being watched in real time in a geographic environment. The flowchart is shown in FIG. 15.

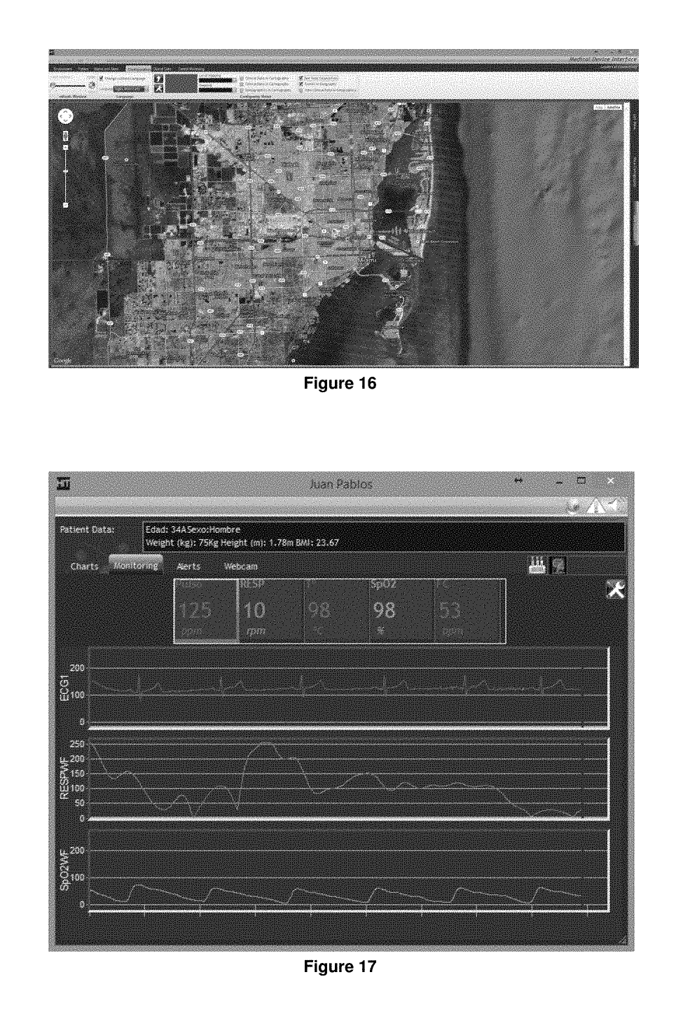

Nurse Jackie accesses the HIT Dashboard module and identifies herself via her user and access password. Next she clicks View Geoposition to access the geographic dashboard (see FIG. 16) which presents a geographic view of the city with the clinic and the patients who are being monitored at home.

She selects the information on patient Juan Pablos who is being monitored at home with a Welch Allyn CVSM device and the HIT Telemonitoring connector of which a photo is attached below. When clicking the patient, a window appears on the geographic map and makes it possible to visualize real-time data capture (see FIG. 17) that is taking place through the abovementioned connector. Data are acquired via the standardization process and transmitted by way of 3G communication to the database that is installed in the clinic.

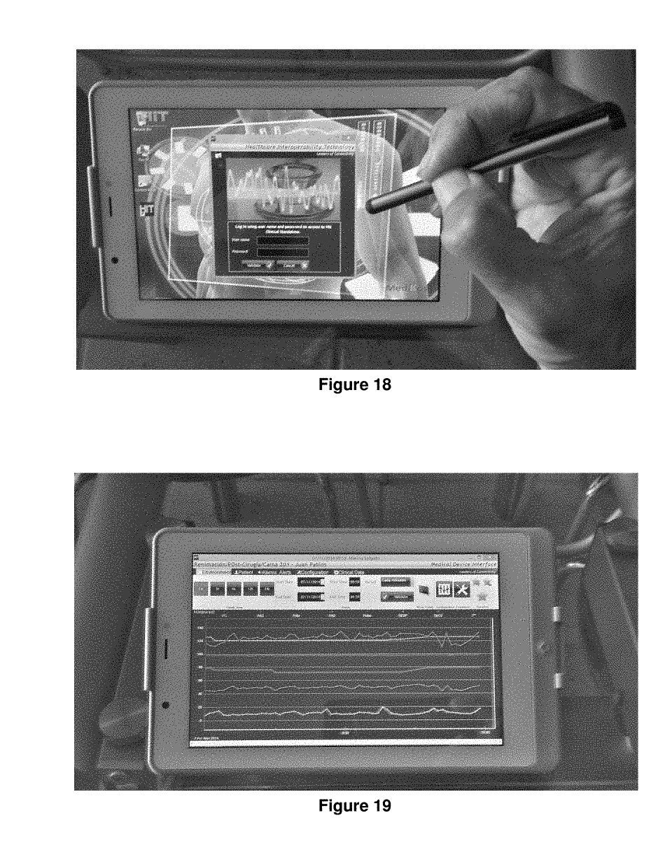

Nurse Jackie realizes that the waveform of ECG derivative 1 is not very normal and she decides to contact Dr. Salazar on the phone to comment about the situation, since he is not at the entity as usual. Dr. Salazar gains access through a portable tablet that has an LTE connection allowing access to the information on patient Juan Pablos. Identification takes place via user and password (see FIG. 18).

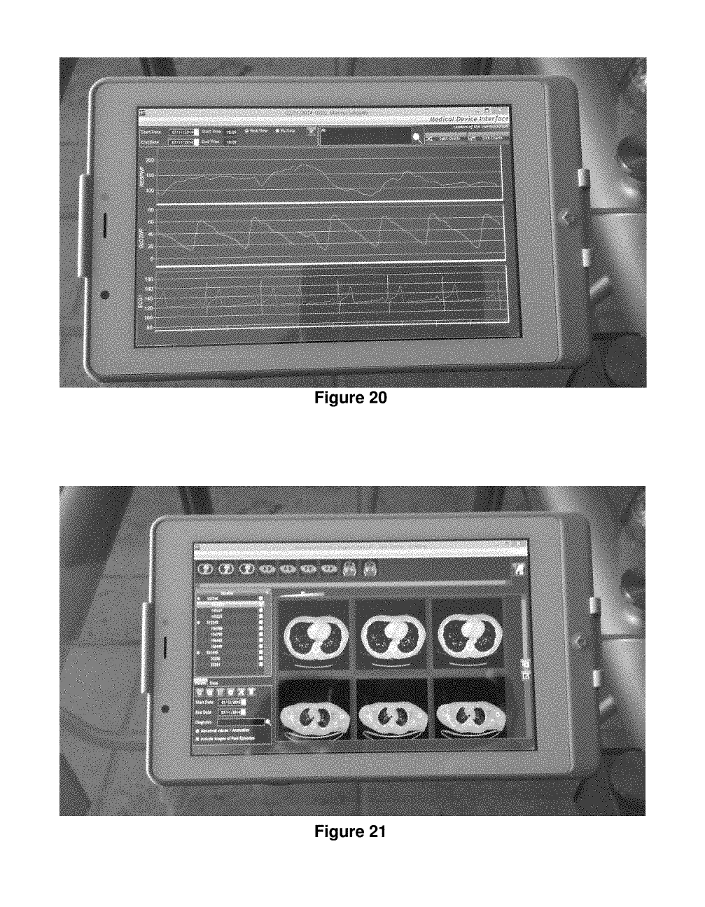

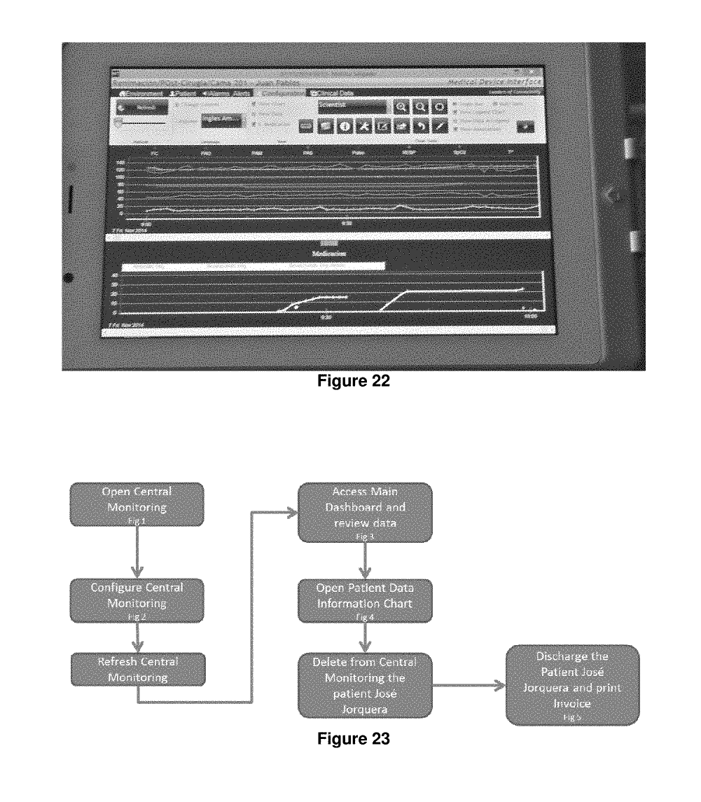

From the list of patients in the HIT Telemonitoring module, Dr. Salazar selects Juan Pablos and accesses information on the last 8 h to see if the graphs are normal (see FIG. 19). In the graph on blood pressure, he watches a drop and decides to check the waveform of the ECG about which he was notified by Nurse Jackie via the button “Waveforms” that is accessed from the monitoring window (see FIG. 20). He reviews the waveform and realizes that there may have been a problem with the pacemaker of the patient. To make sure of it, he clicks “Clinical Data” and checks the images by selecting the icon “Medical Images” that makes it possible to access the system to store radiological images and visualize them in the tablet (see FIG. 21), he zooms in to see the position of the pacemaker better and detects it is correctly implanted, so he immediately decides to check the medication for patient Juan Carlos. To do so, he clicks “Configuration” and selects the option of visualizing the medication for the last 12 h (see FIG. 22), at that moment he realizes that a medication the patient should have taken to stabilize his blood pressure has stopped and that fact is responsible for the waveform Nurse Jackie detected. Dr. Salazar calls Nurse Jackie so that she may notify patient Juan Pablos that he has to retake the medication in order to prevent heart malfunction. Once the communication process is finished, Nurse Jackie can continue Telemonitoring the patient at home via the geographic dashboard.

### Embodiment 4: Central Monitoring of a PACU Unit with Discharge of One of the Patients

At Miami Children Hospital in Miami, Fla., pediatric patients hospitalized at the PICU department are monitored. Monitoring is performed at the entity itself through the Central Monitoring of the product, the activity of the patient in a geographic environment being watched in real time. The flowchart is shown in FIG. 23.

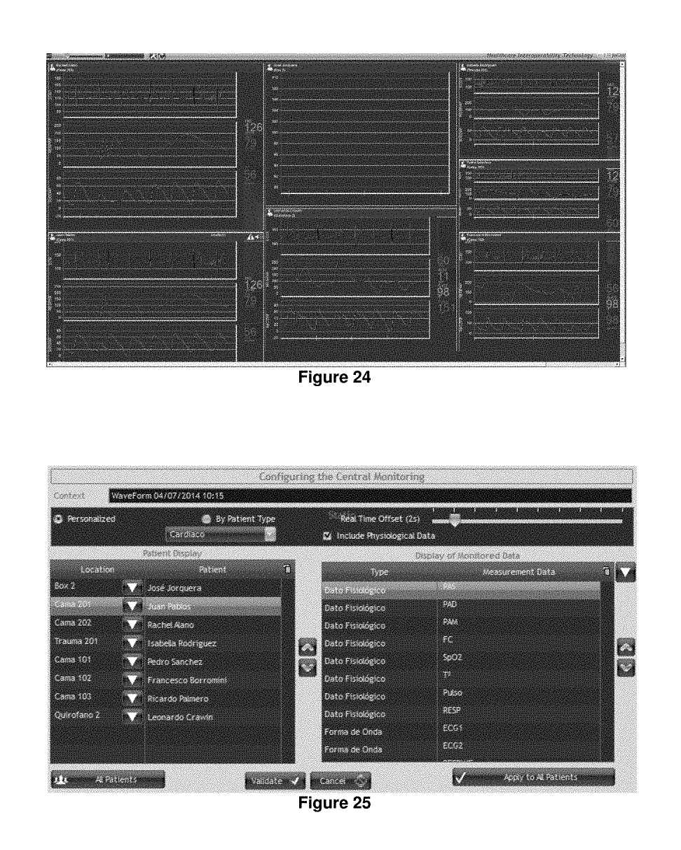

Nurse Susan Mendes starts the session in the HIT Dashboard module to follow up the patients hospitalized at the PICU service. She clicks “Central Monitoring” and selects the Central of the Waveforms type via the respective button. The window containing real-time monitoring of all patients hospitalized in the unit opens (see FIG. 21). In this view, Nurse Mendes can monitor each of the patients independently and with the necessary parameters to follow up the activity of control. This Central Monitoring is composed of widgets making it possible to define the peculiarities of each patient at the level of visualization and they can be positioned freely and resized as the user likes (see FIG. 24).

To define the monitoring parameters of each patient, one must click the icon of configuration, which will open a pop-up window (see FIG. 25) where the patient(s) to be visualized can be selected, each of them will be in an independent widget. The parameters to be visualized (both the weighed physiological data and the signals of waveforms) can be chosen independently for each patient and put in the right order of appearance by using the up and down arrows. In addition, each of them can be deleted by selecting the line and right-clicking, which will delete said parameter or monitoring reading.

The Central Monitoring has a refresh system allowing visualization of data with more or less fluency (from 20 min to 1 s) which is controlled from the slider located on the upper left. This makes it possible to show the data more accurately in real time.

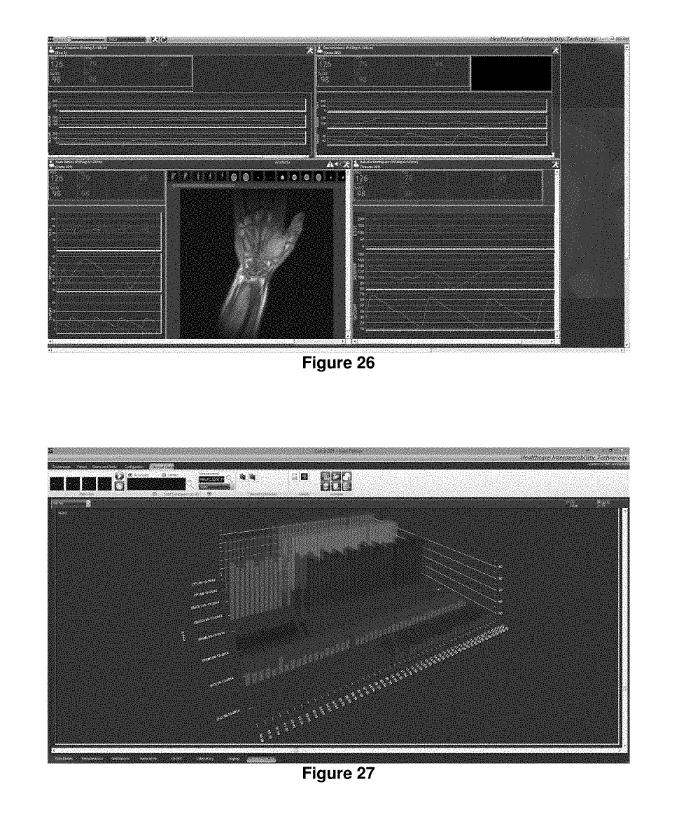

From the same flag on the central screen of HIT Dashboard module, one can launch a much more sophisticated “Control Panel” allowing not only real-time visualization of the data acquired by the different electro medical devices but also visualization of any result from the laboratories and even medical images about the patient in order to have maximum information on the condition of the patient and support decision making. Its functioning is the same as the one described for the Central Monitoring and the graphic part of said dashboard screen is attached in FIG. 26.

From any Central Monitoring, by clicking the icon of the patient that is on the upper left of the widget that contains real-time monitoring, one can access the card containing the evolutionary follow-up of the patient where reading trends, data of lab results, medical images, body water inlets and outlets, administered medication and alarms taking place in the time range selected in “environment” are represented. Nurse Mendes selects the patient Juan Pablos to check his trends for the last two days by applying a 3D view from the comparative flag, where the first axis (X) shows the number of daily hours, ie 24, the second axis shows the data related to each measurement grouped into days so as to see the possible behavioral patterns of the patient, and the third axis shows the value of the parameters or reading performed in an hourly mean (see FIG. 27). This representation makes it possible to cut the time representation in bands of 24 h (or days) in order to analyze if there is any behavioral pattern related to time ranges (morning, afternoon, evening) so as to determine and collate the repercussion of the medication, nursing performances, the sleep, etc. in the health condition of the patient.

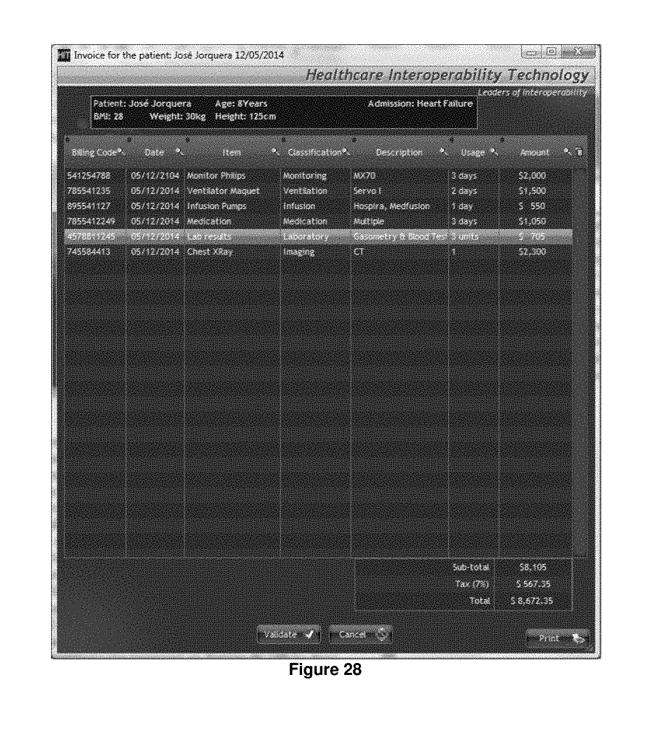

Nurse Susan closes the patient Juan Pablos after validating the information and she realizes that, in the first window of Central Monitoring, the patient Jose Jorquera is not receiving data, she asks her coworkers and they tell her that he has been discharged to the plant. Nurse Mendes immediately selects the flag of patient, selects Jose Jorquera and requests the system to show the discharge in order to recap all data on the patient, including the invoice for the expenses and use of all the material he has been applied (Monitor, Ventilator, Infusion Pumps, Medication, Laboratories, Medical Image) (see FIG. 28). Said invoice is electronically sent to the accounts department of the hospital so that they can record it, the respective billing code being indicated in each of the concepts.

1. A system for a data acquisition from a medical electrical device to acquire, standardize, and/or manipulate a data obtained from medical devices, monitor, and customize each view of the patient, and a real-time visualization of the data, the system comprising:
a first group of connectivity and data management comprising:
a HIT server module (1);
a HIT core module (2);
a second group for visual representation comprising:
a HIT connect module (3);
a HIT dashboard and central Monitoring (4);
a HIT telemonitoring module (5);
a HIT clinical reports module (6);
HIT Advanced Clinical Dashboard (7);
a third group for management and configuration tools comprising:
a HIT Management group including:
a HIT billing module (8);
a HIT management module (9); and
a HIT MD module (10) responsible for the fleet of the medical devices;
a HIT Configuration group including:
a HIT configuration module (11)

2. The system according to claim 1, wherein in said first group of connectivity and data management:
the HIT server module (1) runs in the central server that received the standardized structured data, process it through different statistics and medical algorithm tools; and
the HIT core module (2) runs in the physical connector that is responsible for collecting the data every second or less in real time, structuring the data using a standardization process, creating average minute data using statistical methods, and managing the alarms and alerts data sent by the devices.

3. The system according to claim 1, wherein said modules of said group two clinical viewers display all the acquired and standardized information from the medical devices in addition to external information systems such as laboratory data, EMR, Pharmacy, the clinical viewers comprises:
the HIT connect module (3) is installed in the physical connector and displaying data from the patient associated;
the HIT dashboard and central monitoring module (4) installed in a computer of the facility or entity that has access to a private network and in a HIT Server carrying the information of any patient of the center;
the HIT telemonitoring module (5), a tablet oriented software delivering a home care telemonitoring system following up the health status of patients; and
the HIT clinical reports module (6), a software creating reports based on specific criteria in order to share with the external systems Electronic Medical Records, PHR, and EHR; and
the HIT Advanced Clinical Dashboard (7), a software carrying the historical information of the patient and delivering the patient pathway as a Clinical Data Movie summarizing all the patient progress.

4. The system according to claim 1, wherein in said modules of said third group:
the HIT management module controls the activity of the locations of the entity;
the HIT billing module (8) supports the control of the medical devices used per patient and generates the invoice corresponding to this usage;
the HIT management module (9) focus on statistics and business intelligence analysis; and
the HIT and module (10) is responsible for the fleet of the Medical Devices;
in the second group:
The HIT configuration is use to define the customization of the business rules of the platform and includes a HIT Configuration module (11) having access to the configuration tables of the platform to perform changes on the different parameters and business rules.

5. The system according to claim 2, wherein the HIT Server module (1):
calculates mean son captured values;
produces statistics on acquired values and devices to assess trends selected from the group consisting of mean, minimum and maximum values, range, standard deviation, variance, coefficient, percentage of data out of standard deviation limits, BMI, BSA, and combination thereof;
automatically corrects correlative data caused by external devices;
detects alerts based on user defined criteria;
manages time changes for data storage; and
allows customization of data erasing according to user defined criteria.

6. The system according to claim 2, wherein HIT Core module (2):
standardizes the received data and manages a private or public catalog;
validates the received data according to the intelligence of the system;
establishing priorities and discarding the data coming from less reliable sources; and
univocally identifies the origin of data by the serial number of the device.

7. The system according to claim 3, wherein the HIT connect module (3):
provides a detailed, evolutionary view according to time ranges and shows calculated values such as means or trends;
increases data accuracy according to the selected zooming time;
simultaneously shows clinical data and administration of medication to correlate the effect of drugs or medications with the reaction of the body according to the volume of administered dose;
controls, from the interface, the different devices connected to the patient and controls his different conditions such as localized, connected, and capturing; and
displays the alarms in different user defined formats and classifications and identifies behavioral patterns.

8. The system according to claim 3, wherein the HIT Dashboard and Central Monitoring module (4):
makes it possible to manage an individual series of monitoring per patient, is composed of widgets allowing a very customized composition of the monitoring interface, making it possible to monitor up to 128 patients simultaneously;
allows real-time monitoring of the administration of medication through pumps;
displays 3D clinical data and composes longitudinal graphs in tridimensional graphs with cuts of 24 h or one hour in axis X, the days or hours in axis Y, and the values of the constant(s) in axis Z to assess long-term behavioral patterns.
allows monitoring of inpatients and patients at home; and
allows access to the record of each patient from the central monitoring, making it possible to see the trends, clinical data, or alerts.

9. The system according to claim 3, wherein the HIT telemonitoring module (5) extends the technology to the mobile device such as telephones, tablets, or PDA.

10. The system according to claim 3, wherein the HIT clinical reports module (6):
allows export of clinical reports, generating evolutionary reports that can be differentiated for the use of trend (mean) data and sampling of administration of medication with detection of alerts based on criteria and inventions of the previous modules; and
shows a full report of the medication administered or refused per patient or group of patients, including pumps, manual, dripping, or syringes.

11. The system according to claim 4 wherein the HIT Billing module (8) collates the time of use of devices with the real cost per measurement unit such as sensors used, device modules used, medication and time of performance of the infusion pump of each of them and allows generation of an invoice adjusted to their use that can be integrated into the billing system of the entity, using its billing codes, also allowing assessment of the amortization times of electro medical equipment in the entity.

12. The system according to claim 4, wherein the HIT Management module (9) records the activity per unit of device, identifying each piece of equipment by its serial number, allowing total control on its location, use and functionality; and makes possible to have a statistical database of use per manufacturer, models, and types.

13. The system ents according to claim 4, wherein the HIT md module (10) controls, identifies, and take down mishaps and problems detected in each of the pieces of equipment, supplying reports to those responsible for the technical stock.

14. The system according to claim 4, wherein the HIT Configuration module (11):
allows access to the configuration tables of the platform to perform changes on the different parameters and business rules;
enables creation, modification or deletion of the hospital(s), services, areas, units (bed, boxes) of the facility as well as configuration of the basic parameters of the entity;
handle the users, urofiles and grant access, HIT connectors and servers installed;
catalogue and Master File Management including healthcare standards such as SNOMED, LOINC, DICOM, ICD9 and 10, or Rosetta RTM;
uses 4 active languages at the interface and contents simultaneously at least 7 different languages; and
includes the editor for preventive data analysis based on specific criteria using simple condition operations to detect and notify possible alerts and organ failure risks.

15. A system for visual historical and retrospective representation of data acquired and processed according to claim 3, wherein the module HIT advanced clinical dashboard (7):
allowing to view chronologically all the clinical data using a specific graphical period viewing filter, create medical annotations to share specific important and relative clinical events, bookmark periods of interest that can be shared between physician to go directly to sensitive data, configure all the graphical data displayed including dynamic filtering, and mark alarms on the trends based in minimum and maximum range value according to population standards.
showing a patient history Movie controllable through a simple clinical data player, view detailed information over a specific period of time and manipulate the information through mathematical tools to make measurements on the waveforms, review statistical data on data collected, review clinical events to detect problems and verify its authenticity and avoid artefacts as well as review any Clinical result like labs, medication administration and medical imaging.
